(19) United States
(12) Patent Application Publication
(10) Pub. No.: US 2025/0361650 A1
(43) Pub. Date: Nov. 27, 2025

(54) METHOD OF PROCESSING A RECONSTRUCTED MYCELIUM OBJECT AND A RECONSTRUCTED MYCELIUM OBJECT

(71) Applicant: ECCO Sko A/S, Bredebro (DK)

(72) Inventors: Stojanka Petrusic, Bredebro (DK); Max Gilet, Bredebro (DK)

(73) Assignee: ECCO Sko A/S, Bredebro (DK)

(21) Appl. No.: 18/875,047

(22) PCT Filed: Jun. 15, 2023
(86) PCT No.: PCT/DK2023/050148

§ 371 Date: Dec. 13, 2024

(30) Foreign Application Priority Data
Jun. 15, 2022 (DK) PA 2022 70321

(51) Int. Cl. D01C 1/02; D02G 3/02; D04H 1/4266

(52) U.S. Cl. CPC D01C 1/02; D02G 3/02; D04H 1/4266

(57) ABSTRACT

The invention relates to a method of processing a mycelium object (30) in the form of a mycelium fibril into a mycelium product (MYP), the mycelium object comprising hyphae cells, said hyphae cells having cell walls, the cell walls of said hyphae cells comprising natural polymer including chitin/chitosan polymer, the method of processing the mycelium object (30) includes a reconstruction process (REC) for reconstructing mycelium objects in the form of mycelium fibrils into a mycelium fiber, the method of processing the mycelium object (30) further including at least one of the following processes: a deacetylation process (DEP) a plastification process (PP) a dyeing process (DYP) and a fat liquoring process (FLP).

### FIELD OF THE INVENTION

The invention relates to a method of processing a reconstructed mycelium object according to claim 1.

### BACKGROUND OF THE INVENTION

It is well-known to use mycelium for different types of components such as insulation, materials that resemble a textile or leather, molded items, protective packaging, etc.

A challenge related to mycelium is, however, that the mycelium as such is relatively fragile and delicate, in particular when certain processing criteria are not met. Such criteria may e.g. include water content, flexibility etc. It is an object of the invention to provide a process which in a cost-effective way may provide mycelium end products having desired product properties in relation to e.g. flexibility, softness, visual appearance, strength, etc.

### SUMMARY OF THE INVENTION

The invention relates to a method of processing a mycelium object (30) in the form of a mycelium fibril into a mycelium product (MYP),
the mycelium object comprising hyphae cells, said hyphae cells having cell walls, the cell walls of said hyphae cells comprising natural polymer including chitin/chitosan polymer,
the method of processing the mycelium object (30) includes a reconstruction process (REC) for reconstructing mycelium objects in the form of mycelium fibrils into a mycelium fiber,
the method of processing the mycelium object (30) further including at least one of the following processes:
a deacetylation process (DEP)
a plastification process (PP)
a dyeing process (DYP) and
a fat liquoring process (FLP).

In an embodiment, the mycelium object comprising hyphae cells, said hyphae cells having cell walls, the cell walls of said hyphae cells comprising natural polymer including chitin/chitosan polymer,
wherein at least one of the processes,
the deacetylation process (DEP)
the plastification process (PP)
the dyeing process (DYP) and
the fat liquoring process (FLP)
is performed by subjecting the mycelium object to an exposure of a reactive chemical agent (RCA) and a pressurized processing fluid (PF) in a supercritical condition.

A supercritical fluid able to penetrate a mycelium object can be used as processing medium as long as the selected reactive chemical agents (such as deacetylation, plastification, and dyeing agents) are soluble or transportable in the pressurized fluid.

In this way, mycelium processing can be performed without using large quantities of water and, furthermore, both the processing time and energy consumption are minimized, and the material chemical uptake is maximized. In addition, due to very efficient material penetration, the obtained mycelium product is of good quality with durable and flexible characteristics, and the dyeing quality is homogeneous with a high colour intensity.

In the present context, exposure to the reactive chemical agent and a pressurized processing fluid in a supercritical may typically refer to a mycelium object which has been positioned in process chamber with the reactive chemical agent and where a processing fluid has also been injected into the chamber under pressure to reach supercritical conditions of the processing fluid.

Carbon dioxide is the most widely used supercritical processing fluid because it is a naturally occurring gas and readily available for industrial consumption. Carbon dioxide usually behaves as a gas in air at standard temperature and pressure or as solid when frozen (dry ice). When the temperature and pressure both are increased to be above the critical point (CP) for carbon dioxide, it adopts properties midway between gas and a liquid. Here, it behaves as a supercritical fluid above its critical temperature (31.1° C.) and critical pressure (73.9 bar). In this way supercritical carbon dioxide has liquid-like densities, which is advantageous for mixing with reactive chemical agents, and which, in turn, can help achieve short and effective processing times when compared to using water as the primary processing medium. Unless otherwise noted, the term “processing fluid” refers to carbon dioxide.

Some ethanol could also be used with the SC-CO₂ for a potential drying step. Thus, a supercritical fluid able to penetrate a mycelium object can be used as processing medium as long as the selected reactive chemical agents (such as deacetylation, plastification, and dyeing agents) are soluble or transportable in the pressurized fluid.

A special advantage of the present invention is that fibrils may be processed as individual fibrils, here: mycelium objects, which has been mechanically subdivided from a mycelium precursor, e.g. a mycelium panel. The mycelium fibrils, also referred to as mycelium objects, may also be processed later on in the process towards a mycelium product, e.g. after the mycelium object has been processed into filament, staples fiber, yarn, textile, etc. The great advantages of processing the mycelium objects after being reconstructed is that the reconstructed form is easy to handle in the process as the mycelium object (fibrils) are fixed in the reconstructed fiber. Nevertheless, the processing time may be kept very energy optimized as the very homogenous structure of the reconstructed fiber facilitates shorter and predictable processing times, in particular during supercritical processing.

In an embodiment, the method of processing a mycelium object (30) into a mycelium product (MYP) includes at least one of the processes:
the deacetylation process (DEP)
the plastification process (PP)
the dyeing process (DYP), and wherein the method further includes the
the fat liquoring process (FLP).

In an embodiment, the method of processing a mycelium object (30) into a mycelium product (MYP) includes at least the two processes:
the plastification process (PP)
the dyeing process (DYP), wherein the method further includes the
the fat liquoring process (FLP), and wherein
at least the plastification process (PP) and/or the dyeing process (DYP) is performed by subjecting the mycelium object to an exposure of a reactive chemical agent (RCA) and a pressurized processing fluid (PF) in a supercritical condition,
and wherein the fat liquoring process (FLP) is performed under non-supercritical conditions.

In an embodiment, the exposure of a reactive chemical agent (RCA) and a pressurized processing fluid (PF) in supercritical conditions is performed during a processing time (PTI).

As a part of the process under super critical conditions the pressure of the processing fluid must increase in the beginning of the relevant process. Likewise, the relevant process may end with a pressure reduction, unless a new process is to be performed without reducing the pressure of the processing fluid first. The process of reducing the pressure in the process chamber, e.g. at the end of the processing time, comprises control by a controller in order to reduce the pressure of the pressurized fluid in a controlled manner such that a suitable pressure reduction gradient over a given time is maintained. Controlling the reduction gradient is an advantage for the mycelium objects to maintain desirable characteristics. The reduction period exceeds a time interval of 5 min, such as a time interval between 5 min. to 2 hours, such as 15 min. to 45 min., such as 15 min. to 30 min, such as 30 min. to 2 hours, such as 30 min. to 65 min.

In an embodiment, said mycelium object comprises at least 10% by weight of moisture/water at the time of initiating said subjecting the mycelium object to an exposure of a reactive chemical agent (RCA) and a pressurized processing fluid (PF) in a supercritical state.

According to an advantageous embodiment the water content has to reach an amount that both enables a minimum flexibility of the mycelium before it enters the chamber in order to be processed with e.g. carbon dioxide in a supercritical condition with one or more reactive chemical agents but also to avoid that the mycelium object during processing with supercritical fluid does not dry too much and becomes irreversibly brittle. In other words, the supercritical processing must be performed under process conditions and with an initial water content, which is sufficient to avoid that the processing leads to decrease of strength rather than maintaining the initial strength or even improving the strength. In the present context, strength is to be understood as the degree to which the hyphae cells of the mycelium material attach to one another by chemical bonding such as through covalent bonds, through hydrogen bonds and through Van der Waals forces.

In an embodiment of the invention said mycelium object comprises at least 20% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 30% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 40% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 50% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 60% by weight of moisture/water at the time of initiating said subjecting the mycelium object to an exposure of a reactive chemical agent (RCA) and a pressurized processing fluid (PF) in a supercritical condition.

In an embodiment, at least one of the processes,
the deacetylation process (DEP)
the plastification process (PP)
the dyeing process (DYP),
is performed by subjecting the mycelium object to an exposure of a reactive chemical agent (RCA) and a pressurized processing fluid (PF) in a supercritical condition.

The deacetylation process (DEP), when performed with processing fluid in a supercritical condition, may include that the mycelium object is subjected to a reactive chemical agent such as a deacetylation agent such as a base such as NaOH or to enzymes such as chitin deacetylases (CDAs).

The plastification process (PP), when performed with processing fluid in a supercritical condition, may include that the mycelium object is subjected to a reactive chemical agent such as glutaraldehyde, sulfuric acid, glyoxal, tripolyphosphate (TPP) and/or epichlorohydrin.

The dyeing process (DYP), when performed with processing fluid in a supercritical condition, may include that the mycelium object is subjected to a reactive chemical agent such as dyeing agents such as ionic dyeing agents, such as anionic dyeing agents, such as reactive dyeing agents.

Examples of reactive dyes may include Levafix Brilliant Blue E-BRAN (dye having C.I114 of Dystar Japan Ltd.), Levafix Brill. Red E-RN gran (Dystar Japan Ltd.), Levafix Golden Yellow E-G (dye having C.I27 of Dystar Japan Ltd.), Eriofast RedB (Ciba Specialty Chemicals), Cibacron Red P-BN GRAN (Ciba Specialty Chemicals), Lanasol Red 6G (dye having C.I 84 of Ciba Specialty Chemicals).

In the above three processes, the processing fluid comprises carbon dioxide.

In an embodiment of the invention said mycelium object comprises at least 70% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 80% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 90% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises less than 90% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises less than 85% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises less than 80% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 20% and less than 95% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 40% and less than 90% by weight of moisture/water at the time of initiating said subjecting the mycelium object to an exposure of a reactive chemical agent (RCA) and a pressurized processing fluid (PF) in a supercritical condition.

In an embodiment of the invention said mycelium object comprises at least 60% and less than 92% by weight of moisture/water at the time of initiating said subjecting the mycelium object to an exposure of a reactive chemical agent (RCA) and a pressurized processing fluid (PF) in a supercritical condition.

In an embodiment, the method further includes the process of pre-soaking the mycelium object prior to said exposure of a reactive chemical agent and a processing fluid in a supercritical condition

In an embodiment, the method of processing the mycelium object (30) by a fluid in a supercritical condition into a mycelium product (MYP) includes the following processes:
a plastification process (PP)
a dyeing process (DYP),
a fat liquoring process (FLP) and
a reconstruction process (REC), and wherein at least the following processes are performed in the below consecutive order:
a plastification process (PP)
a dyeing process (DYP),
a fat liquoring process (FLP).

In an embodiment, at least the dyeing process is performed by subjecting the mycelium object to an exposure of a dyeing agent (DYA) and a pressurized processing fluid (PF) in a supercritical condition.

In an embodiment, the dyeing process is performed by subjecting the mycelium object to a dyeing agent (DYA) and a pressurized processing fluid (PF) in a supercritical condition and wherein the plastification process is performed under non-supercritical conditions.

In the present context, the term non-supercritical conditions may refer to a process performed under atmospheric pressure.

In an embodiment, at least the plastification process is performed by subjecting the mycelium object to an exposure of a crosslinking agent (CRA) and a pressurized processing fluid (PF) in a supercritical condition.

In the present context, a plastification process performed by subjecting the mycelium to a crosslinking agent may be referred to as a crosslinking process.

In an embodiment, at least the plastification process (PP) and the dyeing process (DYP) are performed by subjecting the mycelium object to an exposure of a crosslinking agent (CRA), a dyeing agent (DYA) and a pressurized processing fluid (PF) in a supercritical condition at the same time.

In an embodiment, the plastification process (PP) and the dyeing process (DYP) are performed simultaneously by subjecting the mycelium object to a crosslinking agent (CRA) being able to bind covalently to chitosan amine groups, a dyeing agent (DYA) being able to bind to chitin/chitosan hydroxyl groups and a pressurized processing fluid (PF) in a supercritical condition being able to efficiently penetrate the mycelium.

In an embodiment, the plastification agent and the dyeing agent are added to the processing chamber at the same time, thereby enabling both plastification and dyeing at the same time, thereby reducing a need for establishment of two separate supercritical processes, a plastification process and a dyeing process. This may be very advantageous, as the time-consuming process of ramping the pressure up and down may be reduced and also the effective total time, where the processing fluid needs to be in a supercritical condition may be kept as low as possible, thereby also saving energy. Additionally, by utilising different functional groups on the chitin/chitosan polymer for the plastification and dyeing agents, respectively, each chemical process is not limited by the other, and the binding capacity of the chitinous polymer is thus optimized. Furthermore, the predictability of the plastification and dyeing results increases as the risk of binding site competition between the two reactive chemical agents is minimized.

In an embodiment, the soaking process (SOP) includes subjecting the mycelium object to moisture to obtain a moisture content of the mycelium object (30) of at least 10% by weight.

Moisture would in the present context typically refer to water as the dominant moisture component. The water may e.g. be supplemented with detergent.

In an embodiment, said processing fluid (PF) is/comprises carbon dioxide

In an embodiment, the processing time (PTI) is between 15 seconds and 180 minutes.

In an embodiment, the processing time (PTI) is between 15 seconds and 180 minutes at a temperature of above 30 degrees Celsius.

In an embodiment, the chemical reactive agent comprises a crosslinking agent.

In an embodiment, said crosslinking agent is applied to facilitate covalent bonding between mycelium fibrils and/or mycelium fiber(s).

In an embodiment, said crosslinking agent is an aldehyde.

In an embodiment, said crosslinking agent is glutaraldehyde.

In an embodiment, said crosslinking agent is sulfuric acid.

In an embodiment, said crosslinking agent is glyoxal.

In an embodiment, said crosslinking agent is tripolyphosphate (TPP).

In an embodiment, said crosslinking agent is epichlorohydrin.

In an embodiment, the chemical reactive agent comprises a dyeing agent.

In an embodiment, the chemical reactive agent comprises a re-plastification agent. In an embodiment, the chemical reactive agent comprises a base.

In an embodiment, the chemical reactive agent comprises an acid.

In an embodiment, the mycelium object (30) to be processed by the plastification process comprises moisture in an amount of at least 10% to 14% by weight of the mycelium product.

In an embodiment, at least a part of the plastification agent binds covalently to amine groups of the chitosan polymer in the mycelium object during the processing time (PTI).

In an embodiment, the percentage of chitosan amine groups covalently bound to said plastification agent is above 50% such as above 60% such as above 70% such as above 80%.

In an embodiment, the dyeing agent is able to bind covalently to chitin hydroxyl groups.

In an embodiment, the dyeing agent is able to bind covalently to chitosan amine groups.

In an embodiment, the dyeing agent is able to attach mechanically to a mycelium material.

In an embodiment, the dyeing agent is able to react with mycelial cell wall structures by Van der Waals forces.

In an embodiment, the dyeing agent is able to react with mycelial cell wall structures by hydrogen bonds.

In an embodiment, the dyeing agent has functional groups being able to bind covalently to chitin hydroxyl groups, such as ionic functional groups, such as polar functional groups.

In an embodiment, the dyeing agent is an ionic dyeing agent.

In an embodiment, the dyeing agent is an ionic, anionic dyeing agent.

In an embodiment, the dyeing agent is an ionic, anionic, reactive dyeing agent.

In an embodiment, the dyeing agent is water-soluble.

In an embodiment, the mycelium object processed is a filament or yarn reconstructed on the basis of fibrils of chitinous polymer.

In an embodiment, the method includes a plastification process (PP) and/or a dyeing process (DYP) said method comprising the following processes:
providing (2) a mycelium object,
introducing (4) the mycelium object into a process chamber,
subjecting (6) the mycelium object in the process chamber to a pressurized fluid,
controlling (10) the pressure of the pressurized fluid to reach supercritical conditions,
subjecting the mycelium object to a reactive chemical agent, which reactive chemical agent is dissolved and/or transported in the pressurized fluid for at least a predefined period of time while the pressurized fluid is in supercritical conditions,
reducing (12) the pressure in the process chamber,
removing (14) the mycelium object from the process chamber,
wherein the process of reducing the pressure in the process chamber comprises controlling the pressure reduction over time.

In an embodiment, the pressurized fluid comprises CO₂ having a purity of at least 99.0%, preferably at least 99.9%.

The invention further relates to a reconstructed filament wherein the reconstructed filament comprises mycelium fibrils and an additive.

In an embodiment, the reconstructed filament comprises mycelium fibrils and an additive, wherein the reconstructed filament comprises natural mycelium fibrils in an amount of between 70 and 99.9% by weight of the filament and an additive between 0.1 and 30% by weight of the filament.

In an embodiment, the reconstructed filament comprises mycelium fibrils and an additive, wherein the reconstructed filament comprises natural mycelium fibrils in an amount of between 10 and 99.9% by weight of the filament and an additive between 0.1 and 30% by weight of the filament.

In an embodiment, the reconstructed filament comprises mycelium fibrils and an additive, wherein the reconstructed filament comprises natural mycelium fibrils in an amount of between 20 and 99.9% by weight of the filament and an additive between 0.1 and 30% by weight of the filament.

In an embodiment, the reconstructed filament comprises mycelium fibrils and an additive, wherein the reconstructed filament comprises natural mycelium fibrils in an amount of between 30 and 99.9% by weight of the filament and an additive between 0.1 and 30% by weight of the filament.

In an embodiment, the fibrils of the reconstructed fiber consist of mycelium fibrils.

In an embodiment, the mycelium fibrils originate from mechanically subdivided natural mycelium.

In an embodiment, the lengths of the originating mycelium fibers are different from the length of the reconstructed filament.

In an embodiment, the additive(s) comprise(s) at least one of the following: alginate, alginic acid, pectin, carrageenan, carboxymethyl cellulose, starch, polyacrylamide, polyethylene oxide, laccase, transglutaminase, polyphenol, glutaraldehyde, gelatine, casein, glucose, Tris-HCl, phosphate and vinyl acetate.

In an embodiment, the additive comprises at least 100 by weight of carboxymethyl cellulose (CMC) and/or starch.

In an embodiment, the additive comprises at least 100 by weight of carboxymethyl cellulose (CMC) and polyethylene oxide

In an embodiment, the filament comprises at most 85% by weight of the mycelium fibrils, at most 10% by weight the additive carboxymethyl cellulose and at most 5% by weight of the additive polyethylene oxide.

In an embodiment, the filament comprises at most 85% by weight of the natural mycelium fibrils, at most 15% by weight of the additive alginate.

In an embodiment, the breaking strength of the reconstructed filament is at least 6 cN/tex, wherein cN/tex stands for linear mass density-related tenacity.

In an embodiment, the breaking strength of the reconstructed filament is at least 10 cN/tex, wherein cN/tex stands for linear mass density-related tenacity.

In an embodiment, the elongation of the reconstructed filament is at least 5%, wherein elongation is specified as a percentage of the starting length.

In an embodiment, the natural mycelium fibrils comprise plasticized mycelium and comprises a plasticizer, e.g. a crosslinker

In an embodiment, the reconstructed filament is provided according to the method of any of the claims 1-46.

The invention further relates to a reconstructed staple fiber manufactured on the basis of a reconstructed filament according to any of the claims 47-61.

In an embodiment, the natural mycelium fibrils originate from mechanically subdivided natural mycelium material.

In an embodiment, the staple fibers are produced on the basis of said reconstructed filaments.

The invention further relates to a yarn spun from staple fibers of reconstructed filament according to any of the claim 47-61.

The invention further relates to a yarn according to claim 65 comprising a plurality of spun staple fibers based on natural mycelium, wherein the yarn comprises mycelium staple fibers having a length of at least 10 mm.

### THE FIGURES

The invention will now be described in the following with reference to the drawings where

FIG. 1 shows an illustration of a fiber according to the invention comprising fibrous elements,

FIG. 2 illustrates a method for manufacturing a fiber based raw wool according to an embodiment of the invention,

FIG. 3 illustrates a method for manufacturing a fiber based raw wool according to an embodiment of the invention

FIG. 4A-D illustrate different embodiments of the invention,

FIG. 5-10 illustrate examples of an apparatus for processing mycelium objects according to embodiments of the invention,

FIG. 11 illustrates a phase diagram for carbon dioxide,

FIGS. 12a and 12b illustrate an example of a graph of pressure over time, and where

FIG. 13 illustrates an example of a processing method for processing of mycelium objects according to an embodiment of the invention

### DETAILED DESCRIPTION

In this description and claims, the percentage values relating to an amount of material are percentages by weight (wt. %) based on the total weight of the filament or the staple fiber in question unless otherwise indicated. Word “comprising” may be used as an open term, but it also includes the closed term “consisting of”.

In the current application the term “fiber product” refers to fibrous reconstructed filaments or staple fibers.

When referring to natural mycelium material subsequently used for reconstruction, the term “mycelium fiber” can be defined as a single hypha cell.

The term “staple fiber” refers to fibers of discrete length, such as shortened fibrous filaments. The staple fibers include individual fibrils interlocked together. The fibrous filament may be shortened into staple fibers of a certain length.

The staple fibers may be further processed in order to provide an item, such as a yarn or a non-woven material.

The fibrous filaments and/or staple fibers may be used for forming items of footwear. Mycelium based products may also find use within a range of different areas including upholstery, clothing, clothing parts, accessories such as bags, parts of bags, wrist straps, mobile phone covers, etc. Mycelium products may also include parts related to automotive, e.g. textile coverings for seats, textile objects for steering wheel covers, gear knob covers, etc.

Fibrils, the building block of fibers, may be formed of natural fibrous mycelium.

### Fiber Filament

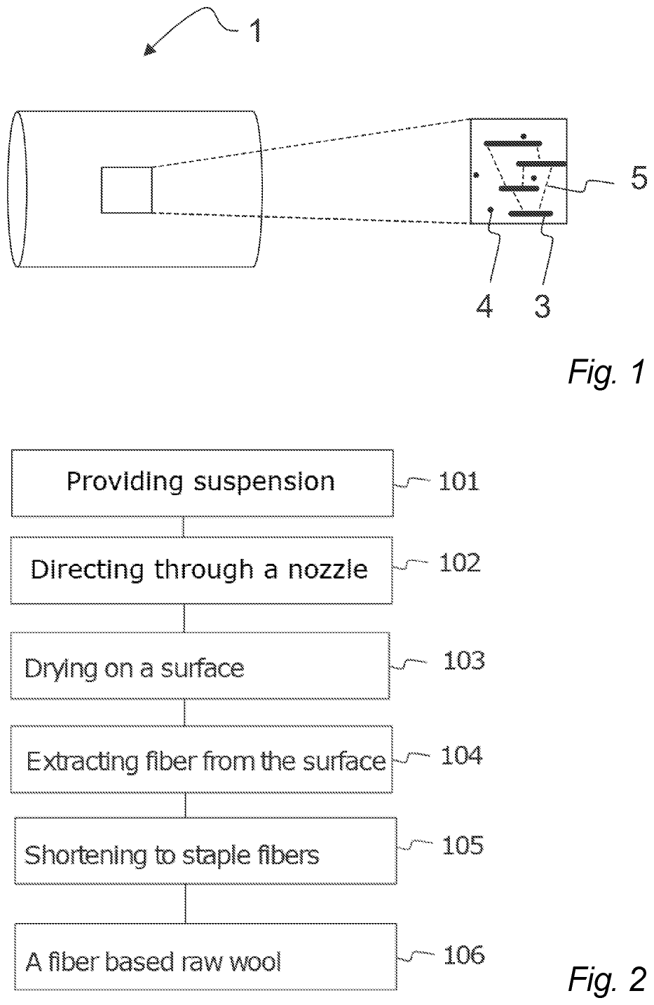

FIG. 1 shows a staple fiber 1 according to an embodiment of the invention

The illustrated staple fiber 1 include fibrous mycelium fibrils 3 in contact with an additive 4. The fibrils 3 are connected to each other, for example via hydrogen bonds 5 and/or mechanical interlocking 5 so as to form a coherent fibrous structure, the staple fiber 1.

The staple fiber 1 may e.g. be formed on the basis of a reconstructed filament based on mycelium fibrils.

### Reconstructed Filament or Staple Fiber

The filament or staple fiber may comprise fibrous elements of fibrils of natural fibrous mycelium.

When using these fibrils for the purpose of reconstructing a filament or a staple fiber on the basis of these, the fibrils may be obtained by mechanical division of the natural fiber source, here mycelium.

In an example a reconstructed filament or a staple fiber comprises fibrous elements of natural fibrous mycelium. In a further embodiment, a reconstructed filament or staple fiber may include mycelium fibrils and on top of that further other types of fibrils, e.g. collagen based fibrils or cellulose based fibrils.

According to an example, a reconstructed filament or a staple fiber product comprises fibrous elements of fibrous mycelium, i.e. fibrils. Preferably, the fibrous mycelium is based on chitin and chitosan polymer and optional further components included in the mycelium as grown and/or further components present in the natural grown mycelium Preferably the fiber product includes at least fibrous elements of mycelium fibrils.

In an example, a reconstructed filament or staple fiber includes at least 50%, for example between 50 and 100%, or between 70 and 100% fibrils which are fibrils of fibrous mycelium. The percentage being given with reference to numbers of fibrils over a certain chosen length.

In an example, a reconstructed fiber product comprises mycelium-based fibrils between 70 and 99.9 wt. %.

In an example, and optionally, a reconstructed fiber product comprises fibrils between 0.01 and 30 wt. % of a further fibril type. Such further fibril type could be cellulosic fibrils originating from other sources than mycelium and/or at least one of the following natural fibrous proteins: Collagen, elastin, keratin, and resilin.

In addition to the fibrils, the reconstructed filament or staple fiber may advantageously include additives. A total advantageous amount of additive(s) may be between 0.01 and 30 wt. %, preferably from 0.05 to 20 wt. % or from 0.1 to 15 wt. %.

Furthermore, the length of the reconstructed staple fibers may in principle made to fit requirements e.g. to a wool made up of the produced staple fibers, the strength or stretch of the finally produced yarn, etc.

### Additives

A reconstructed filament of fiber includes additive(s) like rheology modifier(s), binder(s), cation active reagent(s), crosslinking agent(s), dispersion agent(s), pigment(s), and/or other modifier(s). The applied additives may be designed to fit into the specific application, e.g. considering the natural mycelium source.

In the present context, the additive(s) within the filament/fiber refers to the above additives(s) “internal” function, i.e. facilitating bonding between the individual fibrils of the reconstructed fiber, filament. In other words, a crosslinking is here primarily referring to the crosslinking between fibrils of a reconstructed fiber/filament, whereas crosslinking agent applied for postprocessing of the reconstructed fiber/filament may also facilitate crosslinking between the fibers/filament.

The fibrous filaments and staple fibers may comprise additive(s), such as alginate, alginic acid, pectin, carrageenan, anionic polyacrylamide (APAM), polyethylene oxide (PEO), carboxymethyl cellulose (CMC), starch, enzymes (such as laccase, transglutaminase), polyphenols, glutaraldehyde, gelatine, casein, glucose, Tris-HCl, phosphate or a combination of such.

In an example, the fiber product such as a filament or a staple fiber includes between 0.1 and 30 wt. % or preferably between 0.1 to 15 wt. % additives, which is at least one of the following: alginate, alginic acid, pectin, carrageenan, carboxymethyl cellulose (CMC), starch, anionic polyacrylamide (APAM), cationic polyacrylamide (CPAM), polyethylene oxide (PEO) enzymes (such as laccase, transglutaminase), polyphenols, glutaraldehyde, gelatine, casein, glucose, Tris-HCl, phosphate and resins like vinyl acetate.

According to an example, a reconstructed fiber product such as a filament or a staple fiber includes an additive of carboxymethyl cellulose (CMC) between 0.1 and 15 wt. %.

According to an example, a reconstructed fiber product such as a filament or a staple fiber includes 85 wt. % of fibrils of mycelium and total amount 15% of additives. The additives may be as follows: 10 wt. % of carboxymethyl cellulose (CMC) and 5 wt. % of polyethylene oxide (PEO).

During manufacturing of the reconstructed fiber product, such as a filament or a staple fiber, the additive, such as an alginate, may have effect on forming hydrogel. In addition, alginate may act as a binder in the fiber product structure. Alginate may cause crosslinking, which may have effect on binding of fibrils of the fiber product. Alginate matrix may crosslink around the fibrils and enclose the fibrils. In an example, the fiber product, such as fibrous filaments and staple fibers, may comprise between 0.1 and 15 wt. % of binder, such as alginate.

The reconstructed fibrous filaments and staple fibers may comprise a crosslinking agent and reagent pair. A crosslinking agent may be arranged to react with the reagent at the nozzle exit. Crosslinking reaction between the crosslinking agent and reagent pair creates an aqueous hydrogel and thereby influences the initial strength of the fibrous suspension. A fiber product may comprise 0-25 wt. %, preferably between 0.5 and 25% or between 1 and 10 wt. % of the crosslinking agent. The crosslinking may have effect on improving the properties of the reconstructed fiber, such as wet and/or dry strength. It may also have effect on stretch and washability of the fiber product.

The fibrous filaments and staple fibers may comprise a dispersion agent. A dispersion agent may comprise anionic long chained polymer, carboxymethyl cellulose (CMC), starch, anionic polyacrylamides (APAM), cationic polyacrylamide (CPAM), or a combination of such. A fibrous filament may comprise 0-20 wt. %, preferably between 0.5 and 25% or between 1 and 10 wt. % of a dispersion agent. The dispersion agent may have effect on shear strength of the fiber product. In an example, the fiber product may comprise between 0.1 and 10 wt. % of additives such as CMC (carboxymethyl cellulose) and/or starch.

### Manufacturing of a Reconstructed Filament or Staple Fiber

A fibrous suspension is provided. The term “suspension” refers here to a heterogeneous fluid containing solid particles, such as fibrils. It encompasses also slurries and dispersions. Typically, solid particles are in aqueous liquid. The fibrous suspension comprises an aqueous suspension of natural fibrous polymers, such as fibrils of fibrous mycelium.

Fibrous elements, i.e. fibrils, of mycelium may be provided through mechanical disintegration of the mycelium raw material, such as material comprising mycelium hyphae. The mycelium is mechanically divided so as to form mycelium fibrils depending on the application. Mechanical disintegration refers to any mechanical way of disintegration or fibrillation of mycelium material to obtain mycelium fibrils. Fibrillation may generally within the scope of the invention be carried out, for example, using a stone mill, refiner, grinder, homogenizer, colloider, supermass colloider, friction grinder, ultrasound-sonicator, fluidizer, such as microfluidizer, macrofluidizer or fluidizer-type homogenizer. In an example, mycelium fibril suspension may be refined using Masuko grinder.

Prior to the mechanical disintegration, also referred to as mechanical dividing, the mycelium being the source of the fibrils may be chemically treated, e.g. by using enzymes reducing the energy consumption of the subsequent process.

The suspension may comprise water, fibrils of natural mycelium, and at least one additive. The additive may include or be a mycelium bonding agent facilitating e.g. hydrogen bonding between the mycelium fibrils and/or other types of bonding, such as covalent bonding.

The fibrous suspension is directed through a nozzle so as to form a fibrous filament. The nozzle may feed the fibrous suspension to a surface. The surface may be a surface of a belt or of a cylinder. The fibrous suspension is dried on the surface. Drying removes water from the fibrous suspension. The dried fibrous suspension is arranged to form a fibrous filament onto the surface. The fibrous filament may be continuous. The continuous fibrous filament may be extracted from the surface. The fibrous filament extracted from the surface may be cut or shortened in order to form staple fibers.

Alternatively, the fibrous suspension fed to the surface is arranged to be shortened and dried on the surface. The dried and shortened fibrous suspension is arranged to form staple fibers. This is enabled by surface structure, such as grooves arranged on a curved surface. The shortened fibers are extracted from the surface.

Mycelium objects may also refer to objects containing mycelium parts, such as filament or yarn reconstructed from fungal mycelium according to the processes e.g. as disclosed in PCT/EP2018/053849, PCT/EP2018/053848, now with the use of” fungal cellulose” (mycelial chitin/chitosan polymer) as the primary material source instead of collagen. These referred documents refer to processing of collagen and optionally also cellulose, but in the present context, the same process may be applied on chitin/chitosan. Such yarn or filament may thus be understood as a mycelium object within the scope of the invention, as long as the small mycelium parts such as individual chitin/chitosan polymers, also referred to as fibrils, originate from fungal mycelium, even if the mycelium object in such case also includes an additive promoting the gathering of mycelium fibrils, e.g. by bonding.

FIG. 2 illustrates a method for manufacturing a natural fiber based raw wool according to an embodiment. An alternative within the scope of the invention is to directly extrude reconstructed filament and wind it up on a spool A mycelium suspension is provided 101. The mycelium suspension comprises aqueous suspension of mechanically divided mycelium fibrils. In other words, the mycelium fibrils as such are to be regarded as natural fibril. The mycelium suspension may comprise water, divided mycelium fibrils and at least one rheology modifier. Fibrils of the mycelium suspension may originate from mechanically subdivided mycelium material.

The mycelium suspension is directed through a nozzle 102. The nozzle feeds the mycelium suspension to a surface. The surface may be a surface of a belt or of a cylinder. The mycelium suspension is dried on the surface 103. Drying removes water from the mycelium suspension. The dried mycelium suspension is arranged to form a reconstructed fiber on the surface. The reconstructed fiber may be arranged in a form of a continuous reconstructed fiber. The continuous reconstructed fiber is extracted from the surface 104. The reconstructed fiber extracted from the surface is cut or shortened in order to form staple fibers 105. The stable fibers are arranged to form an inhomogeneous network comprising fiber concentrations of varying density and orientation. The inhomogeneous fluffy material of staple fibers is called a natural fiber based raw wool 106.

The above process is described as a process using natural mycelium material as a source for the manufacture of staple fibers.

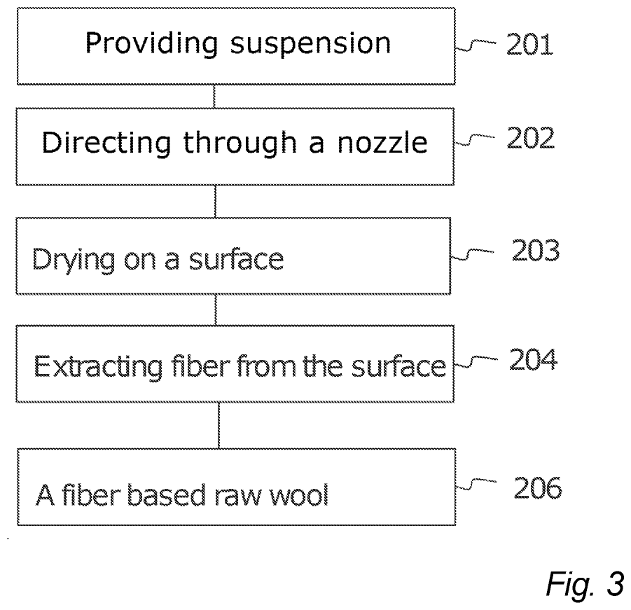

FIG. 3 illustrates a method for manufacturing a natural fiber based raw wool according to an embodiment. A mycelium suspension is provided 201. The mycelium suspension comprises aqueous suspension of divided mycelium fibrils. The mycelium suspension may comprise water, at least one rheology modifier and divided mycelium fibrils. Divided mycelium fibrils may originate from plasticized (e.g. cross-linked) mycelium precursor MYPK, which has been mechanically subdivided into pulp. The mycelium suspension is directed through a nozzle 202. The nozzle feeds the mycelium suspension to a surface, for example on a surface of a belt or of a cylinder. The mycelium suspension is arranged to be shortened and dried on the surface 203. The dried and shortened mycelium suspension is arranged to form staple fibers. This is enabled by grooves (not shown) arranged on a surface. In following embodiments grooves refer to structures enabling a formation of staple fibers having a length defined by the distance between the grooves. Those grooves, including grooves 401 and 601 may generally alternatively be made as ridges performing the same function, i.e. weakening strength of the reconstructed filament at positions where the filament may divide or break into the desired reconstructed staple fibers. The shortened fibers are extracted from the surface 204. The stable fibers are arranged to form an inhomogeneous network comprising fiber concentrations of varying density and orientation. The inhomogeneous fluffy material of staple fibers is called a natural fiber based raw wool 206.

Mechanical disintegration into mycelium fibrils from mycelium raw material, mycelium pulp, or refined pulp is carried out with suitable equipment such as a refiner, grinder, homogenizer, colloider, friction grinder, ultrasound sonicator, fluidizer such as microfluidizer, macrofluidizer or fluidizer-type homogenizer.

Mycelium fibers may be isolated from any relevant mycelium containing raw material using chemical-, mechanical-, bio-, thermo-mechanical-, or chemi-thermo-mechanical pulping process. Mechanically shortened, divided or cut fibers may comprise chemically or physically modified derivative of mycelium micro fibrils or fibril bundles.

A mycelium suspension may comprise 80-98 wt-% of water and 2-20 wt-% of mycelium. The mycelium suspension may comprise 85-98 wt-% of water and 2-15 wt % of mycelium. In addition, the mycelium suspension may comprise 0-5 wt-% of rheology modifier.

A mycelium suspension may comprise 80-98 wt-% of water and 2-20 wt-% of mycelium. The mycelium suspension may comprise 85-98 wt-% of water and 2-15 wt % of mycelium. In addition, the mycelium suspension may comprise 0-5 wt-% of rheology modifier.

The mechanically divided mycelium fibril may be pure mycelium structures or comprise chemically modified or chemically treated mycelium fibrils. Thus, the mycelium suspension comprises mechanically divided or shortened mycelium fibers.

Fiber product may include additives like rheology modifier(s), binder(s), cation active reagent(s), crosslinking agent(s), dispersion agent(s), pigment(s), and/or other modifier(s).

Fiber and staple fibers may comprise additives such as alginate, alginic acid, pectin, carrageenan, anionic polyacrylamide (APAM), cationic polyacrylamide (CPAM), polyethylene oxide (PEO), carboxymethyl cellulose (CMC), starch, enzymes (such as laccase, transglutaminase), polyphenols, glutaraldehyde, gelatine, casein, glucose, Tris-HCl, phosphate or a combination of such.

Rheology modifier comprises a compound or agent arranged to modify the viscosity, yield stress and/or thixotropy of the suspension. Rheology modifier may comprise high molecular weight polymers. Rheology modifier is arranged to modify mycelium suspension rheology by adjusting gel strength and yield point of the mycelium suspension.

### Mycelium Object

In the present context, the term “mycelium object” refers to any form or collection of mycelium including a mycelium precursor, a mycelium fibril or any mycelium-based intermediate in the process

As mentioned elsewhere in the present application, the term “fungal mycelium precursor material” MYPRE (in short “mycelium precursor”) refers to an undifferentiated and untreated fungal mycelium network comprising hyphae cells. By letting the mycelium precursor react with one or more plastification agents, a robust and stabile “fungal mycelium intermediate material” (in short “mycelium intermediate”) arises. The mycelium intermediate is then suitable for being dyed, dried and compressed into a final “fungal mycelium product” (in short “mycelium product”) resembling leather or other textile materials. The order of which the mycelium intermediate is processed (plastified, softened, dyed, dried), etc. may vary, although exemplified advantageous examples are given in the present application. However, when the mycelium object has been reconstructed into a filament, a staple fiber, a textile product, etc and the mycelium and/or object has been plasticized properly to give the final product the desired properties, the object is then referred to as a mycelium product MYP
is compacted into a static and irreversible material, it will be referred to as a “compact fungal mycelium material” (in short “compact mycelium”) irrespective of whether e.g. drying, dyeing and/or other processing elements have been performed.

It is however noted that a compression in the present context may be omitted or performed with a very moderate pressure, e.g. simply in order to press moisture out of the mycelium.

Mycelium is the vegetative part of a fungus, i.e. not including fruitbodies, comprising a mass of branching, thread-like hyphae cells. The mass of hyphae is sometimes called shiro, especially within the fairy ring fungi. Fungal colonies composed of mycelium are found in and on soil and many other substrates. A typical single spore germinates into a monokaryotic mycelium, which cannot reproduce sexually; when two compatible monokaryotic mycelia join and form a dikaryotic mycelium, that mycelium may form fruitbodies such as mushrooms. A mycelium may be minute, forming a colony that is too small to see, or may grow to span thousands of acres as in Armillaria. Through the mycelium, a fungus absorbs nutrients from its environment. It does this in a two-stage process: First, the hyphae cells secrete enzymes onto or into the food source, which break down biological polymers into smaller units such as monomers. These monomers are then absorbed into the mycelium by facilitated diffusion and active transport.

Mycelia are vital in terrestrial and aquatic ecosystems for their role in the decomposition of plant material. They contribute to the organic fraction of soil, and their growth releases carbon dioxide back into the atmosphere. Ectomycorrhizal extramatrical mycelium, as well as the mycelium of arbuscular mycorrhizal fungi, increase the efficiency of water and nutrient absorption of most plants and confers resistance to some plant pathogens. Mycelium is an important food source for many soil invertebrates. They are vital to agriculture and are important to almost all species of plants many species co-evolving with the fungi. Mycelium is a primary factor in a plant's health, nutrient intake, and growth, with mycelium being a major factor to plant fitness.

The mycelium processes addressed in the present application may be understood as part of the mycelium processing which happens after the mycelium has harvested and when the living organisms in the fungus has preferably been killed.

The cell wall of a mycelium hyphae cell is a three-layered structure comprising chitin, glucans and mannoproteins. In order to modify and stabilize the molecular structure of mycelium, the cell wall glucans and mannoproteins may be removed to access the chitin polymer, which is the part of hyphae having the highest reactive potential. Chitin molecules are complex carbohydrate polymers containing N-acetylglucosamine and N-glucosamine monomers. Naturally occurring fungal chitin is thus not a true homopolymer but exists as a copolymer comprising units of both chitin and its deacetylated homologue chitosan. The chitosan level of a mycelium can be defined as the degree of chitin deacetylation (DD) which, in turn, can be calculated as the number of glucosamine (GlcN) units divided by the total number of glucosamine and acetyl glucosamine (GlcNAc) units:

$$DD ( \% ) = \frac{G l c N}{( G l cN + GlcNAc )} \cdot 100$$

When DD is at least 50%, the name of the polymer shifts from ‘chitin’ to ‘chitosan.’ In naturally occurring fungi, the deacetylation degree is generally low, and the cell wall carbohydrate polymers are therefore referred to as chitin.

In the context of mycelium processing for the purpose of producing leather (-like) textiles, the chitin/chitosan polymer of the fungal cell wall may be of high importance. In the present context, the terms “chitinous polymer”, “chitosaneous polymer”, “chitin/chitosan polymer”, “chitin/chitosan molecule”, “fungal cellulose”, “mycelial fibril” and “mycelium fibril” are used interchangeably meaning a “complex carbohydrate polymer of the fungal cell wall comprising N-acetylglucosamine and N-glucosamine monomers wherein said complex carbohydrate polymer has a deacetylation degree between 0 and 100”.

To obtain a durable, flexible and firm mycelial textile material, chemical bonding between individual chitin/chitosan polymers may advantageously occur. This can be achieved by the addition of bridging molecules such as crosslinking agents to the mycelium. However, chitin does not have readily reactive functional groups and is insoluble in most solvents thus limiting its industrial application potential. To overcome this challenge, a deacetylation reaction can be introduced in order to convert chitin into its deacetylated homologue chitosan. Chitosan has readily reactive functional amino groups which are able to form amide bonds with different crosslinking agents. Amide bonds resist hydrolysis and confer structural rigidity at the molecular level. Furthermore, chitosan is soluble in dilute acidic solutions and the industrial application potential thus increases when chitin deacetylation takes place. It should be noted that even though covalent bonding between crosslinking agents and mycelium cell wall chitosan is an advantageous bridging technique, other, less rigid, ways of plasticizing fungal mycelium could be achievable as well.

In the present context, crosslinking agents could include glutaraldehyde, sulfuric acid, glyoxal, tripolyphosphate (TPP) and/or epichlorohydrin.

Thus, in an advantageous embodiment, a chitin deacetylation process is included in order to establish particularly favourable conditions for the intended subsequent plastification process PP, of the fungal mycelium precursor material. A deacetylation process can be facilitated by different means, e.g. by chemical or enzymatic treatment of the mycelium.

In the present context, plastification can be defined as a process in which a fungal mycelium strengthening is obtained by the introduction of bridging molecules between e.g. individual chitin/chitosan polymers in the hyphae cell walls. Attachment of such bridging molecules to the mycelium can occur through covalent bonding or through other chemical reactions such as by hydrogen bonding, through Van der Waals forces, or e.g. by mechanical trapping. Plastification can potentially also occur without bridging molecules, e.g. through direct interaction between individual chitin/chitosan polymers.

Furthermore, in an advantageous embodiment of the invention, the hydroxyl groups of the chitin/chitosan molecule may react with appropriate dyeing agents, and this reaction may occur at the same time as the plastification reaction takes place. As the two reactive chemical agents (plastification and dyeing agents) target different functional groups of the chitin/chitosan polymer, the reactions may occur in a so-called “one pot” manner. It should be noted that the dyeing and plastification processes are not limited to occur simultaneously, and that dyeing agents may also be able to interact with other functional groups and through other chemical reactions than by covalent bonding between dyeing agent and chitin/chitosan hydroxyl groups. Appropriate dyeing agents include acid dyes, sulfur dyes, direct dyes, premetallized dyes, reactive dyes and/or basic dyes.

In an advantageous embodiment, plastification is performed by the use of chemical crosslinking agents. During such process, the addition of chemical crosslinking agents redirects the inter- and intramolecular hydrogen bonds of the chitin/chitosan polymer and creates covalent bonds with the amine groups of chitosan. This ultimately changes and stabilizes the properties and characteristics of the overall mycelium material. Treating the material with an acidic solution after the primary crosslinking reaction has taken place can in some cases help fixate the agents to the chitosan binding sites. The flexibility and softness of the material may be further increased by the introduction of fat liquoring agents which can create protective and lubricating helix structures around the individual mycelium hyphae.

In the present context, a “reactive chemical agent” refers to any chemical substance which, when exposed to a mycelium object, reacts chemically (e.g. by covalent bonding, by hydrogen bonding, through Van der Waals forces, by mechanical trapping etc.) with biochemical structures within the mycelium hyphae network. Thus, reactive chemical agents include, but are not limited to, deacetylation agents, plastification agents, e.g. crosslinking agents, and/or dyeing agents.

A supercritical fluid is able to efficiently penetrate a mycelium structure and can replace water as processing medium as long as the selected reactive chemical agents are soluble or transportable in the pressurized fluid. In this way, mycelium processing can be performed without using large quantities of water and, furthermore, both the processing time and energy consumption are minimized.

In the present context, pressurized fluids represent compounds which adopts properties midway between a gas and a liquid and behaves as a supercritical fluid.

Any substance is characterized by a critical point which is obtained at specific conditions of pressure and temperature. When a compound in a liquid state is subjected to a pressure and a temperature higher than its critical point, the fluid is said to be “supercritical”.

Carbon dioxide is the most widely used supercritical fluid because it is a naturally occurring gas and readily available for industrial consumption.

Carbon dioxide usually behaves as a gas in air at standard temperature and pressure or as a solid when frozen (dry ice). When the temperature and pressure both are increased to be above the critical point (CP) for carbon dioxide, CO₂ adopts properties midway between a gas and a liquid. Here, it behaves as a supercritical fluid above its critical temperature (31.1° C.) and critical pressure (73.9 bar). In this way, supercritical carbon dioxide has liquid-like densities, which is advantageous for dissolving and transporting reactive chemical agents. Furthermore, the gas-like low viscosities and diffusion properties of a supercritical fluid can facilitate shorter reaction times and better uptake compared to water.

The critical point of a pressurized fluid may vary according to various conditions such as e.g. the density and/or purity of the fluid. The method for processing mycelium objects may therefore not only be possible in a supercritical condition but also in near-supercritical conditions. Supercritical and near-supercritical conditions may be used interchangeably in the present context. Thus, it should be understood that when, in the claims and description of the present application, reference is made to “pressurized fluid in the supercritical condition” or similar terms, such terms will include a pressurized fluid that is in a near-supercritical condition.

The term “supercritical carbon dioxide” or “SC-CO₂” may be used interchangeably in the present context. Also, carbon dioxide and CO₂ may be used interchangeably in the present context.

In the present context, the term “fungal mycelium precursor material” (in short “mycelium precursor”) refers to an undifferentiated and untreated fungal mycelium network comprising hyphae cells. By letting the mycelium precursor react with one or more plastification agents, a robust and stabile “fungal mycelium intermediate material” (in short “mycelium intermediate”) arises. The mycelium intermediate is then suitable for being dyed, dried and compressed into a final “fungal mycelium product” (in short “mycelium product”) resembling leather or other textile materials. The order of which the mycelium intermediate is processed (e.g. dyed, plasticized, dried, or compressed) is not fixed and may vary. Advantageous sequences are however disclosed in the present application. However, when the mycelium object is compacted into a static and irreversible material, it will be referred to as a “compact fungal mycelium material” (in short “compact mycelium”) irrespective of whether e.g. drying, dyeing and/or other processing elements have been performed.

Thus, the procedure of compression/compacting can in principle be performed at any processing stage between the plastification process and the mycelium product.

The term “mycelium object” refers to any of the above-mentioned descriptions of mycelium material irrespective of its processing status.

The term “dye” or “dyeing” is in the present context referring to the addition of dyeing substances to the mycelium object with the purpose of obtaining a desired colour. Such dyeing process within the scope of the invention would preferably be performed at supercritical carbon dioxide conditions.

In an advantageous embodiment, dyeing agents comprise acid dyes, direct dyes and/or reactive dyes which can bind covalently or attach mechanically to the mycelium material, or react with mycelial surface structures by Van der Waals forces or by hydrogen bonding. In some cases, dyes can even react with previously introduced chemicals within the mycelium material though advantageously, biochemical dyeing processes result in covalent bonding between dyeing agents and functional groups of the chitin/chitosan polymer.

The process of dyeing may be performed in a process chamber under supercritical conditions, but generally, it should be noted that the dyeing process may be applied with any suitable dyeing equipment designed to dye according to the provisions of the invention.

The term “fat liquoring” refers to the process where fats/oils and waxes are fixed to the mycelium material. In more detail, the degree of chitin/chitosan polymer cohesion is decreased by the addition of oils or fats as a fat liquoring agent is able to cause a detachment of a mycelial fiber (hypha) from its neighboring fiber. However, as some degree of fiber cohesion needs to be present in order to keep the material in a coherent shape, the addition of oils or fats needs to be performed in a controlled manner.

Any fat liquoring agent may be used, including anionic fat liquors such as sulfonated fat liquors and sulfited oils, soap fat liquors and cationic fat liquors. Nonionic fat liquors may also be used, including alkyl ethylene oxide condensates and protein emulsifiers. Multicharged fat liquors that are formulations of non-ionic, anionic and cationic fat liquors, may also be used for the fat liquoring process.

Raw material for the fat liquoring agents may be sea animal oils such as fish oil; land animal oils and fats such as claw oil, beef tallow, pig fat and bone fat; vegetable oils and fats such as palm oil, sunflower oil, rapeseed oil, soybean oil, coconut fat, palm kernel fat and turkey red oil; waxes such as carnauba wax, montan wax and wool grease; synthetic fats such as paraffin oil, mineral oil, fatty alcohol and fatty acid ester.

Thus, examples of fat liquoring agents may be sulfated oils as well as raw oils and waxes.

In a preferred embodiment, fat liquoring agents (fats, oils and waxes) for the processing of fungal mycelium originate from non-animal sources.

The mycelium objects may be processed into finalized mycelium products.

Mycelium products may include pre-cut parts for e.g. a shoe, where such parts could e.g. be a vamp, toe cap, tongue, quarter or a heel cap. Mycelium products may also include products where the mycelium object has been laminated with other layers, e.g. reinforcement layers, where the mycelium products has been stretched across the surface of a rigid form, e.g. a smartphone casing, e.g. accessories which was typically made by or on the basis of leather, etc.

Thus, mycelium products may find use within a range of different areas including upholstery, clothing, clothing parts, accessories such as bags, parts of bags, wrist straps, mobile phone covers, etc. Mycelium products may also include parts related to automotive, e.g. textile coverings for seats, textile objects for steering wheel covers, gear knob covers, etc.

Mycelium objects may also refer to objects containing mycelium parts, such as filament or yarn reconstructed from fungal mycelium according to the processes e.g. as disclosed in PCT/EP2018/053849, PCT/EP2018/053848, now with the use of” fungal cellulose” (mycelial chitin/chitosan polymer) as the primary material source instead of collagen. These referred documents refer to processing of collagen and optionally also cellulose, but in the present context, the same process may be applied on chitin/chitosan. Such yarn or filament may thus be understood as a mycelium object within the scope of the invention, as long as the small mycelium parts such as individual chitin/chitosan polymers, also referred to as fibrils, originate from fungal mycelium, even if the mycelium object in such case also includes an additive promoting the gathering of mycelium fibrils.

It should be noted that a “mycelium object” being processed within the scope of the invention, may mean that one, two or more mycelium objects may be processed at the same time.

As used herein, “at least one” is intended to mean one or more, i.e. 1, 2, 3, 4, 5, 6, 7, 8, 9, 10, etc.

The word “comprising” may be used as an open term, but it also includes the closed term “consisting of”.

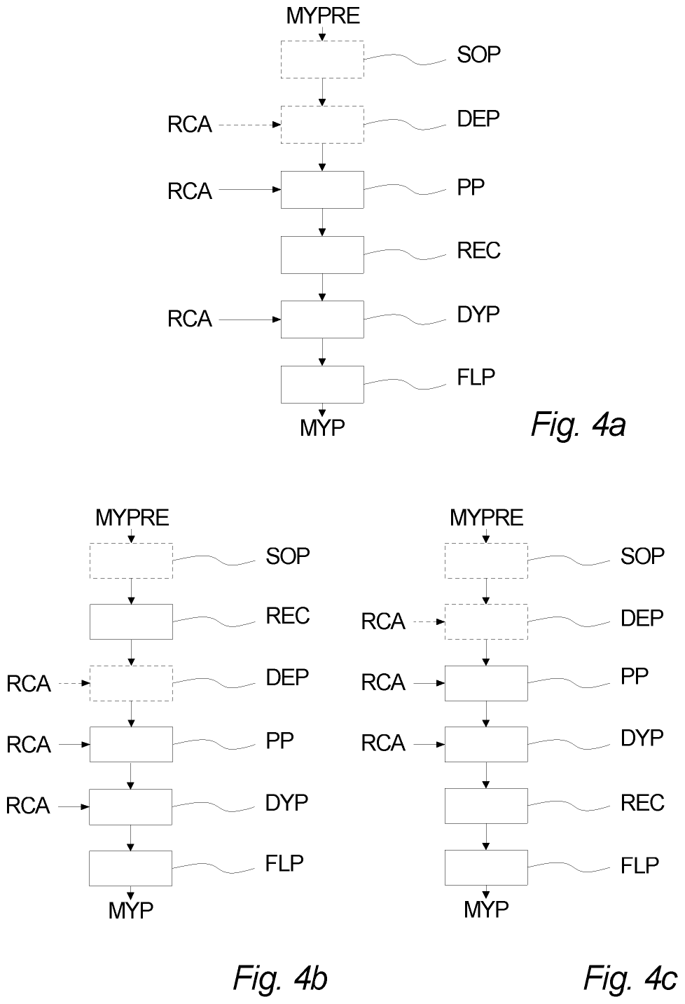

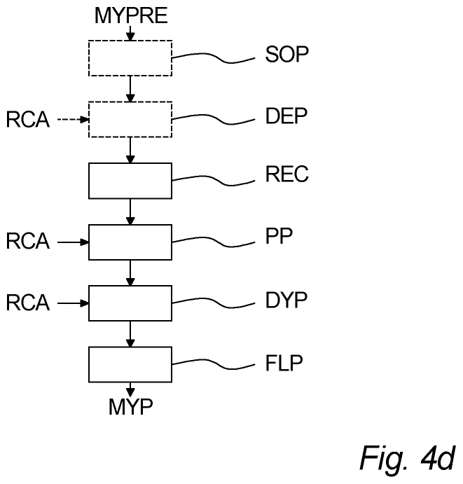

FIG. 4A-D illustrate different possible workflows for processing a mycelium object starting from a mycelium precursor MYPRE and, finally, turning it into a (final or more or less final) mycelium product MYP. The order of the individual processes may vary in different embodiments of the invention and thus, the individual processes may be performed in an order different from the ones presented and illustrated in the figures. Other variants of the method may also be applied within the scope of the invention. However, still keeping this flexibility broadly in mind, certain processes may or should advantageously be performed in a certain order. This will be discussed below.

For each workflow diagram presented, one of the processes may be run under the influence of a supercritical fluid such as supercritical CO₂ (carbon dioxide). This could also include more than one process, a combination of processes or, potentially, all of the presented processes.

To obtain a textile/leather-like mycelium product, the mycelium object may go through a drying process which could be introduced at any time during the overall process. During drying, the water content of the mycelium object is actively decreased either inside or outside the supercritical fluid environment. If drying is performed inside the supercritical fluid environment, a moisture absorbent may be introduced. To prevent the material of becoming brittle and lose its flexibility and resistance, the drying should be only partial such that the final mycelium product contains at least 10% moisture.

A method of processing a mycelium object will now be explained with reference to FIG. 4A.

During a first process, a soaking process, SOP, processing fluid is made to flow into the mycelium precursor MYPRE and saturate (or nearly saturate) it with such processing fluid, here water. The purpose of the soaking process is to penetrate the mycelium precursor with processing fluid to obtain a uniform moisture content throughout the interior of the mycelium precursor thereby facilitating subsequent processing of the mycelium object. It may be advantageous that the soaking process takes place prior to deacetylation, plastification, dyeing and fat liquoring, as soaking of the mycelium precursor may facilitate a more uniform and predictable modification of the material. It should be noted that the first process, the soaking process SOP, is optional insofar the mycelium precursor already has an acceptable water content. According to an advantageous embodiment, the water content should be not too high, but also be high enough to ensure that the desired chemical processes are able to run, but also to ensure that the processes to be run in a supercritical condition do not contain too much water.

In a further process, a deacetylation process, DEP, an optional deacetylation reaction takes place. Here, the mycelium precursor is treated with a deacetylation agent in order to obtain a higher deacetylation degree of the chitin/chitosan polymers within the fungal mycelium. This process prepares the mycelium precursor for subsequent processing such as plastification, by increasing the number of readily reactive functional groups to which the plastification agents can bind. Deacetylation can be performed by different means such as by enzymatic treatment or by alkaline treatment. The process is obtained through use of a reactive chemical agent RCA, indicated in the drawing.

A further process is a plastification process PP, in which long chain molecules such as chitinous polymers of the fungal cell wall are brought to react with chemical bridging molecules to link such individual long chain molecules to one another. Attachment of bridging molecules to the mycelium can occur through covalent bonding, i.e. crosslinking, or through other chemical reactions such as by hydrogen bonding, through Van der Waals forces, or by mechanical trapping/blocking. Plastification can potentially also occur without bridging molecules, i.e. through direct interaction between individual chitin/chitosan polymers.

The process is obtained through the use of a reactive chemical agent, RCA, as indicated in the drawing. The plastification process strengthens, stabilizes and softens the mycelium, e.g. by the introduction of covalent bonds between individual chitin/chitosan molecules, thereby converting the fungal mycelium precursor material into a fungal mycelium intermediate material. Crosslinking can be performed by using different reactive chemical agent(s), here crosslinking agents, such as glutaraldehyde, sulfuric acid, glyoxal, tripolyphosphate (TPP) and/or epichlorohydrin. In an advantageous embodiment, reactive chemical agents used for the mycelium plastification process, PP, are crosslinking agents such as aldehydes.

At this point, a reconstruction process REC is performed on the basis of the plasticized mycelium.

This process basically implies that the plasticized mycelium material is mechanically subdivided, e.g. by a grinding process, into mycelium fibrils and the mycelium fibrils are then reconstructed into a mycelium fiber, filament or staple fiber.

The subsequent process steps are thus performed on the reconstructed fiber.

A further process is an optional re-plastification process (not shown) which may be applied after the main plastification process, PP, on the reconstructed fiber, in order to add further strength and secure material uniformity. Here, the mycelium intermediate is retreated with plastification agents by reusing different crosslinking agents such as glutaraldehyde, sulfuric acid, glyoxal, tripolyphosphate (TPP) and/or epichlorohydrin.

In a further process, a dyeing process DYP, the mycelium intermediate, a reconstructed mycelium fiber, is treated with reactive chemical agents, here dyeing agent(s), such as acid dyes, direct dyes and/or reactive dyes, in order to obtain a desired coloration of the material.

In a further process, a fat liquoring process, FLP, the mycelium intermediate, the reconstructed mycelium fiber, is exposed to fat liquoring agents such as sulfonated fat liquors or sulfited oils, soap fat liquors or cationic fat liquors.

A further method of processing a mycelium object will now be explained with reference to FIG. 4B.

This method corresponds largely to the method illustrated in FIG. 4A, but now with a reconstruction process REC performed already between the soaking process SOP and the deacetylation process DEP.

The deacetylation process DEP and the subsequent process steps are thus performed on the reconstructed fiber.

The dyeing process DYP is here run under supercritical conditions, but the dyeing process may also be performed with conventional dyeing measures under atmospheric conditions.

A further method of processing a mycelium object will now be explained with reference to FIG. 4C.

This method corresponds largely to the method illustrated in FIG. 4A, but now with a reconstruction process REC performed between the dyeing process DYP and the fat liquoring process FLP.

The fat liquoring process FLP is thus performed on the reconstructed mycelium fiber.

The dyeing process DYP and the fat liquoring process FLP may be run under supercritical conditions. This has the advantage that the fat liquoring process FLP may be assisted and run more efficiently than a fat liquoring process run under atmospheric pressure.

A further method of processing a mycelium object will now be explained with reference to FIG. 4D.

This method corresponds largely to the method illustrated in FIG. 4A, but now with a reconstruction process REC performed between the deacetylation process DEP and the plastification process PP.

The plastification process PP and the subsequent process steps are thus performed on the reconstructed fiber.

It should be noted that the application of supercritical fluid during processing of a mycelium object may be applied with a special advantage in relation to dyeing and/or plastification, but that the apparatus and method described herein also may be applied for other processes, e.g. the deacetylation process DEP, of the methods illustrated in FIG. 4A-D. A specific example related to a dyeing process is explained and this process may be incorporated into e.g. the above illustrated process flows 4A-D as a suitable dyeing process run under supercritical conditions.

To facilitate an optimal flow of reactive chemical agents through the mycelium object, said mycelium object may be moist when entering the supercritical fluid environment.

More precisely, the mycelium material may be in a condition where its maximum water-absorption capacity is met without the material being over-saturated thereby spontaneously losing water. Regarding the surrounding gaseous medium of the supercritical fluid environment, the relative humidity may be 97% or higher, such as 98% or higher, such as 99% or higher. In an example, the relative humidity in the environment of the reaction chamber and the material should be equal for optimal processing, e.g. dyeing.

Hence, the exemplary process steps of FIG. 4a-d may be varied and configured in different way within the scope the invention and only serves for the purpose of enabling a skilled person to implement the present invention in one or few specific ways out of many.

The above implementations of FIG. 4a-d and variations thereof may be performed with conventional process equipment such as open or closed containers in manual or automatic work processes where relevant process compounds are applied to the mycelium fibrils and/or fibers at feasible temperatures. In other words, these embodiments will not apply supercritical processes.

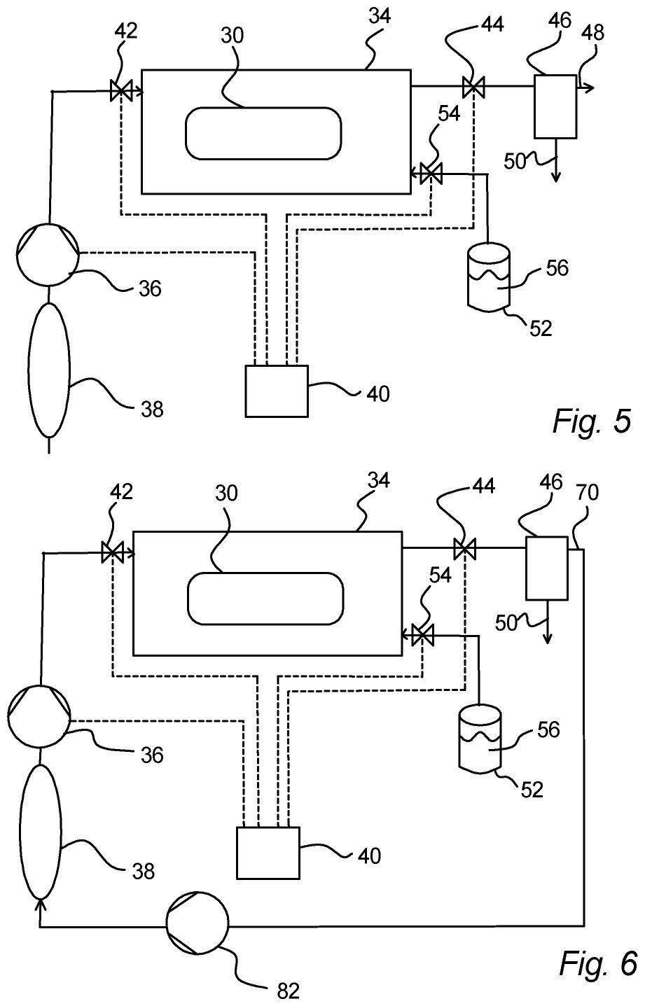

In further advantageous embodiments of the invention, the above implementations of FIG. 4a-d and variations thereof may be performed wholly or partly under supercritical conditions in any of the more advanced exemplified apparatuses described and explained in the below FIGS. 5-10.

FIG. 5 illustrates an example of a mycelium textile process in an embodiment of the invention. A mycelium object 30 is processed in a process chamber 34.

The mycelium object 30 is in the present context referring to one of a plurality of mycelium-based fibrils located in the process chamber. In reality, although only one mycelium object is specifically referred to in this and the below examples, it will always be accompanied by a plurality of other mycelium objects (mycelium fibrils), whether the fibrils are loose or whether the fibrils have been reconstructed into a filament, staple fiber, textile etc. Unless otherwise stated in the below FIG. 5-10, this meaning will be implied.

The process chamber may be configured in the form of a pressure chamber. The pressure chamber may be in connection to at least one controllable compressor 36 for pressurizing a fluid, which is supplied from a storage container 38 e.g. a high pressure storage container. The pressure provided by the controllable compressor 36 is introduced to the pressure chamber 34 by an introducing member 42, e.g. a controllable valve or the like. At an output end, the pressure can be reduced by a pressure reducing member 44, e.g. in the form of a controllable valve, pressure reducing valve or the like. The apparatus according to an embodiment of the invention, may also comprise a separator 46, which receives the escaping pressurized fluid and where for example residue dyeing agent may be separated from the pressurized fluid. The pressurized fluid can leave the separator 46 via an outlet 48, and the separated residue dyeing agent may be collected via a residue outlet 50. A reactive chemical agent 56 is introduced from the source of reactive chemical agent 52 into the process chamber via a controllable inlet 54, e.g. a controllable valve or the like, and in an embodiment of the invention, the reactive chemical agent is a dye. The introduction and release of pressure and introduction of dye are controlled by a controller 40, where the controller 40 as illustrated may be connected to the controllable compressor 36, the introducing member 42, the controllable inlet 54 and the pressure reducing member 44 to control these in dependence on such parameters as time, pressure, temperature, characteristics of the mycelium object, etc. Furthermore, the control may also be performed in relation to circulation flow rate, speed of the movable process chamber, stirrer etc. Thus, it will be understood that the apparatus may be equipped with corresponding sensors, e.g. pressure sensors, temperature sensors, etc. and that sensor signals may be communicated to the controller 40 as further input for the control of the apparatus.

The pressurized processing fluid may be circulated and reused from the pressurized chamber. The pressurized processing fluid may also or as an individual process be circulated and reused after leaving the separator and via a recirculation compressor lead back into the high-pressure storage container.

FIG. 6 illustrates an example of an apparatus in an embodiment of the invention as described in FIG. 5. FIG. 6 further comprises that the pressurized processing fluid may be circulated and reused after leaving the separator 46 with a recirculation connection 70 and a recirculation compressor 82 leading back into the storage container 38.

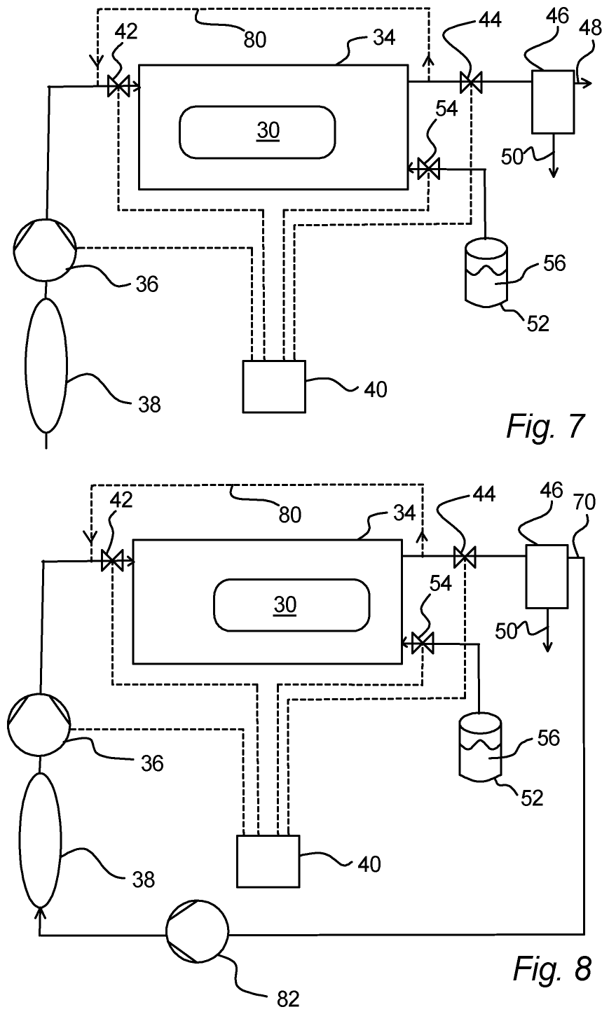

FIG. 7 illustrates an example of an apparatus in an embodiment of the invention as described in FIG. 5. FIG. 7 further comprises that the pressurized processing fluid may be circulated and reused from the pressurized chamber back into the pressurized chamber 34 as illustrated with the recirculation connection 80.

FIG. 8 illustrates an example of an apparatus in an embodiment of the invention as described in FIG. 5. FIG. 8 further comprises that the pressurized processing fluid may be circulated and reused after leaving the separator 46 with a recirculation connection 70 and a recirculation compressor 82 leading back into the storage container 38 and that the pressurized processing fluid may be circulated and reused from the pressurized chamber back into the pressurized chamber 34 as illustrated with the recirculation connection 80.

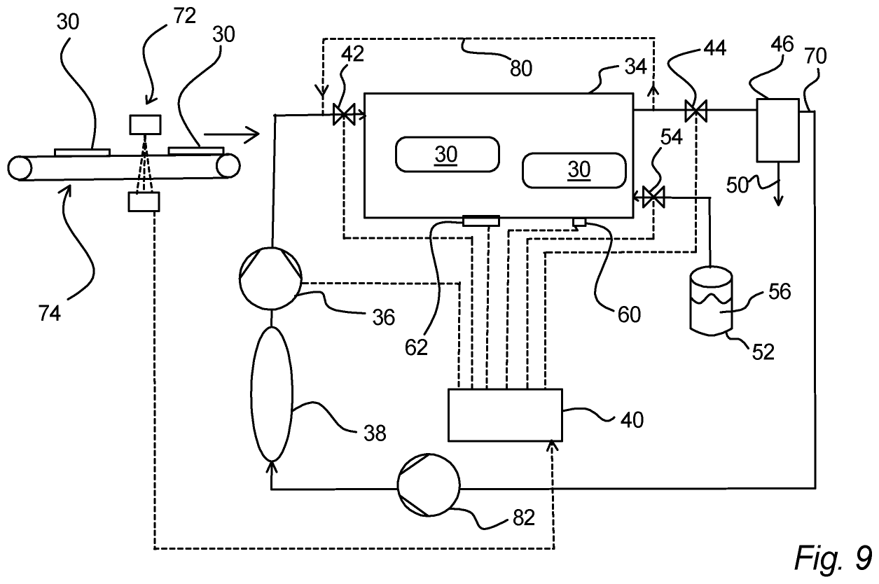

FIG. 9 illustrates further possible embodiments of an apparatus according to the invention. The apparatus as shown in FIG. 9 corresponds essentially to the example shown in FIG. 8, but wherein it is further indicated that a temperature sensor 60 can be arranged within or in connection with the process chamber 34 in order to measure the temperature of the fluid, e.g. the supercritical fluid. The measured temperature is communicated to the controller 40 and based on e.g. controller software control signals which may be communicated to a heater and/or cooler 62 in order to achieve a desired temperature of the fluid. It should be noted that the desired temperature of the fluid may depend on the pressure of the fluid and that a pressure sensor (not shown) may be arranged as well. Alternatively, a measure for the pressure of the pressurized fluid may be given by the controllable compressor 36 or the introducing member 42.

Furthermore, it is illustrated in FIG. 9 that two or more mycelium objects 30 may be processed in the process chamber 34 at the same time.

As mentioned in relation to FIG. 5, a mycelium object 30 will be associated by other mycelium fibrils. The meaning of indicating two mycelium objects 30 in the present context, is to explain that the two indicated mycelium objects may be gathered with other mycelium fibrils in two different “gathering”, e.g. being a filament, staple fiber, textile, etc.

Even further, it is illustrated in FIG. 9 that the mycelium objects 30 may be supplied to the apparatus in an automated manner, for example with mycelium objects 30 being supplied to the process chamber 34 at least partly via a schematically shown supply 74 of mycelium objects 30, which for example may be a conveyer, a conveyor belt or the like. In connection with such a supply of mycelium objects 30, mass, thickness and/or volume detector means 72 may be arranged in order to determine mycelium characteristics that are of importance to the supercritical process. Such mass, thickness and/or volume detector means 72 may for example comprise a radiation detection apparatus as indicated in FIG. 8, but other apparatus features such as weighing cells, video monitoring and analysis, etc. may be used as well. The determined mass, thickness and/or volume of the mycelium objects may be communicated to the controller 40, which on the basis hereof may determine a corresponding amount of e.g. reactive chemical agent to be added to the fluid in the process chamber 34, when the specific mycelium object is to be processed, and the controller 40 may communicate this to the controllable inlet 54, and possibly e.g. to the source of reactive chemical agent e.g. by a weight scale or volume scale. It should be noted that other parameters of the mycelium objects 30 may be provided by the detector means and used instead or in addition as input to the controller 40 for determining the necessary amount of reactive chemical agent. Such other parameters may be the surface area, the mycelium object type, e.g. texture or the like.

It should furthermore be noted that the mycelium objects 30 may be supplied in bulk to the apparatus and that they may be processed in bulk, e.g. with the weight of two or more of the mycelium objects 30 being provided as a bulk parameter and with the mycelium objects of the bulk being processed, e.g. dyed, at the same time.

Pressurized fluid may be in liquid form but may also be in gas form.

The mycelium object 30 may be a whole piece of mycelium without any pre-cutting or trimming or may be a piece of mycelium, e.g. a mycelium object that has been through a cutting or trimming process. There may also be more than one piece of mycelium materials included in the overall process.

Reactive chemical agent may be introduced to the process chamber at the same time as the mycelium object but may also already be present in the chamber before or introduced after the mycelium object is subjected to the chamber.

The process chamber can have any form relevant for mediating the optimal conditions to maintain supercritical conditions over time. In embodiments of the invention, the process chamber and apparatus may in some relations appear in small scales and in other relations in big scales depending on the given applications.

Also, it should be noted that the process chamber 34 may comprise means for agitating the mycelium object(s) and the supercritical fluid in relation to each other, e.g. drum rotating means, rotating object carriers, a stirrer, etc. or other arrangements involving movement of the process chamber or parts thereof, but the apparatus may instead or in addition comprise e.g. pumping means for circulation of the supercritical fluid. The control of the process chamber may further comprise settings such as speed, direction movement etc.

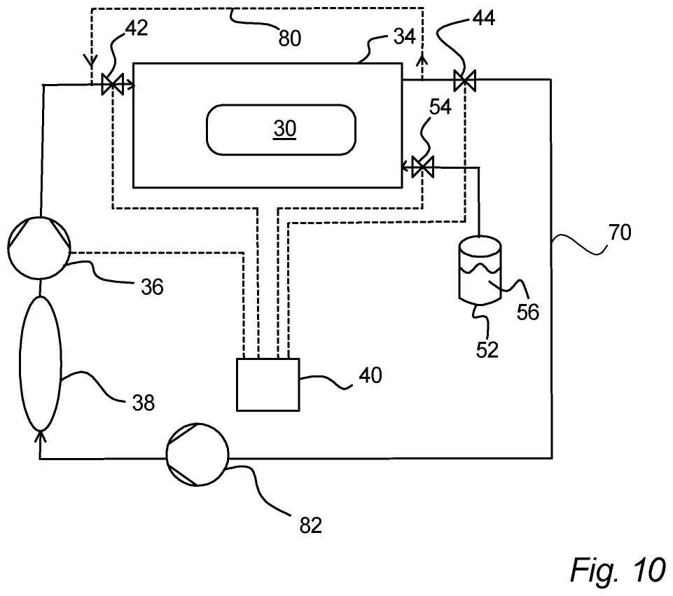

FIG. 10 illustrates a further possible embodiment of an apparatus according to the invention. The apparatus as shown in FIG. 10 corresponds essentially to the example shown in FIG. 8, but the modification that will be explained in the following can be implemented in any other of the embodiments that are described herein. As described in connection with FIG. 8, the pressurized processing fluid may after the process, where the supercritical fluid and the reactive chemical agent are being circulated via the recirculation connection 80, leave the process chamber via the separator 46. However, as indicated in FIG. 10, the separator has been omitted and the separation of surplus dye can instead be performed within the process chamber 34, e.g. by reducing the pressure of the processing fluid, whereby any residue will be separated from the fluid and eventually fall to the bottom of the process chamber. When the surplus reactive chemical agent has been separated, the processing fluid, e.g. CO₂, can be e.g. pumped from the process chamber 34 via the reducing member 44, via the recirculation connection 70 and the recirculation compressor 82. Hereby, the processing fluid will be led back into the storage container 38. As regards the residue reactive chemical agent in the process chamber 34, this can be collected and/or a rinsing cycle can be made with e.g. CO₂ in order to clean the process chamber and its connections.

It should be noted that a rinsing cycle may be used as well in connection with other embodiments disclosed in the present application.

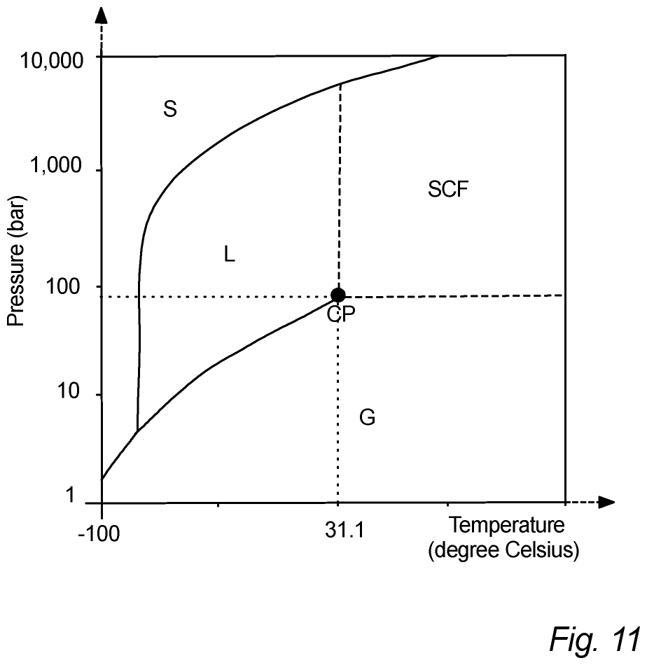

FIG. 11 illustrates a scale phase diagram for carbon dioxide (schematic and not to scale). Carbon dioxide behaves as a gas G in air at standard temperature and pressure or as solid S when frozen. When the temperature and pressure both are increased to be above the critical point CP for carbon dioxide, it adopts properties midway between a gas and a liquid L. Here, it behaves as a supercritical fluid SCF above its critical temperature (31.1° C.) and critical pressure (73.9 bar).

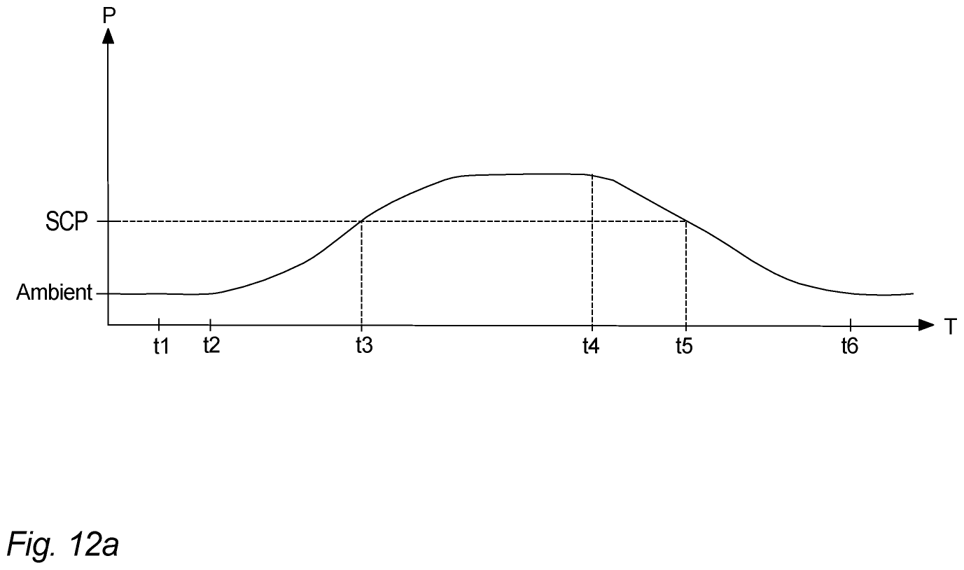

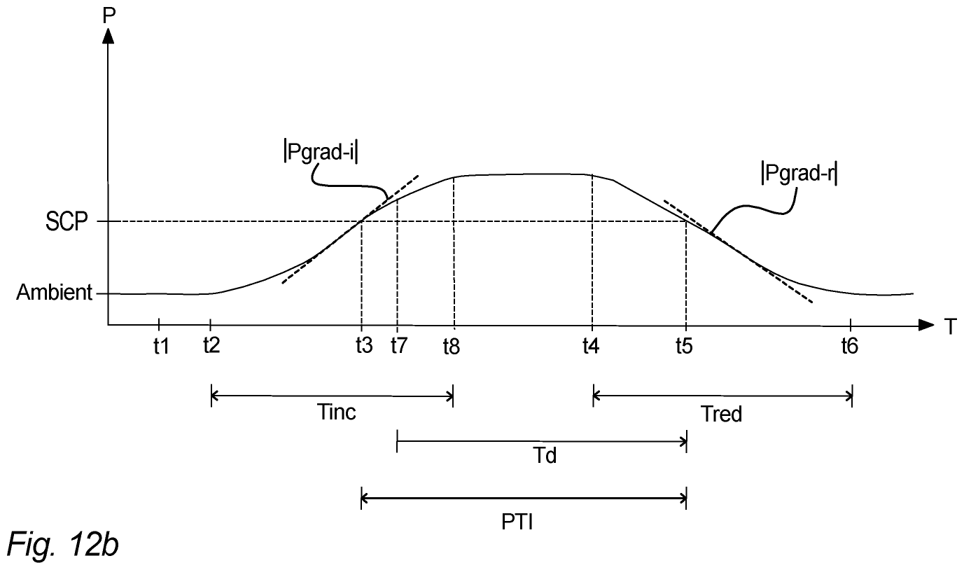

FIG. 12a illustrates an exemplified timeline of the pressure P over time T, e.g. illustrating the condition in the process chamber 34 during a mycelium object processing cycle. The pressure may start at ambient A pressure t1 and at this point a mycelium object may be introduced into the process chamber. After a given time, the pressure is increased t2, e.g. by introducing and further pressurizing a pressurized processing fluid such as CO₂ and increases until a critical point of pressure CP t3. The gradient of the increase of pressure over time may be a steep increase wherein the pressure is increased over a shorter period of time, or the increase may also be slower wherein the pressure is increased slower over a given time. Between t3 and t5, supercritical conditions are kept over a given time. The illustrated curve is in this example showed with a flat top with a constant pressure over time, however, the top could also have a pressure increase over time extending directly into a decrease without having a constant pressure over time. After a given period of time t4, the pressure is decreased and decreases over time until ambient conditions are reached. The gradient of the decrease of pressure over time may be a steep decrease wherein the pressure is decreased over a shorter period of time, or the decrease may also be slower wherein the pressure is decreased slower over a given time.

Reactive chemical agent may be added to the process chamber in the beginning of the process e.g. at t1 or t2 but may also be added later maybe during the supercritical conditions. Possible excess reactive chemical agent may be released and removed from the process chamber (or separated from the pressurized processing fluid leaving the process chamber) when the pressure decreases or when conditions have reached ambient conditions.

FIG. 12b illustrates a corresponding exemplified timeline of the pressure P over time T, wherein the essentially same pressure curve and the same points of time are shown as in FIG. 12a. Furthermore, it is illustrated as an example in FIG. 8b that reactive chemical agent is added to the process chamber at the time t7, i.e. after the processing fluid has reached supercritical conditions. Consequently, as shown below at the time axis (T-axis), the mycelium objects in the process chamber will be subjected to reactive chemical agent dissolved or diluted in the supercritical fluid for a period Td corresponding to t5-t7.

A processing time PTI is indicated between t3 and t5, where the process conditions are supercritical.

Further, it is illustrated in FIG. 12b that the pressure reaches a maximum value at t8, whereafter the pressure remains essentially constant until t4. Thus, as shown below at the time axis (T-axis), the mycelium objects in the fluid will be subjected to a pressure increase for a period Tinc corresponding to t8-t2. Also, it is shown that the pressure gradient may be determined and monitored, here indicated as the numerical value IPgrad-iI. The apparatus may be configured to control the pressure increase by monitoring the period Tinc, which must exceed a predefined increase period such as e.g. 15 min. such as e.g. 25 min., such as e.g. 30 min. such as between 5 min. to 1 hour, such as 15 min. to 45 min., such as 15 min. to 30 min., or the apparatus may be configured to control the pressure increase by monitoring the pressure gradient, e.g. the numerical value IPgrad-iI. which must not exceed a predefined pressure increase gradient such as e.g. 20 bar/min, such as 15 bar/min, such as 10 bar/min, such as 8 bar/min, such as 6 bar/min, such as 5 bar/min or such as 4 bar/min.

Even further, as illustrated in FIG. 12b below the time axis (T-axis), the mycelium objects in the processing fluid will be subjected to a pressure reduction for a period Tred corresponding to t6-t4. Also, it is shown that the pressure gradient may be determined and monitored, here indicated as the numerical value IPgrad-rI. The apparatus may be configured to control the pressure reduction by monitoring the period Tred, which must exceed a predefined reduction period such as e.g. 15 min. such as e.g. 25 min., such as e.g. 30 min such as between 5 min. to 2 hours, such as 15 min. to 45 min., such as 15 min. to 30 min, such as 30 min. to 2 hours, such as 30 min. to 65 min., or the apparatus may be configured to control the pressure reduction by monitoring the pressure gradient, e.g. the numerical value IPgrad-rI., which must not exceed a predefined pressure reduction gradient such as e.g. 10 bar/min, such as 8 bar/min, such as 6 bar/min or such as 4 bar/min.

The diagram is schematic and time intervals for pressurization and reduction may vary from each other, even considerably, and pressurization may be much faster than depressurization, thus meaning that the curve may be relatively steeper for the pressurization.

It should be noted that FIG. 12a og b refers to panels of mycelium prior to subsequent fibrilization, i.e. processing a mycelium panel or the like into mycelium fibrils. The same process on fibrilized mycelium may advantageously be performed with a shorter processing time

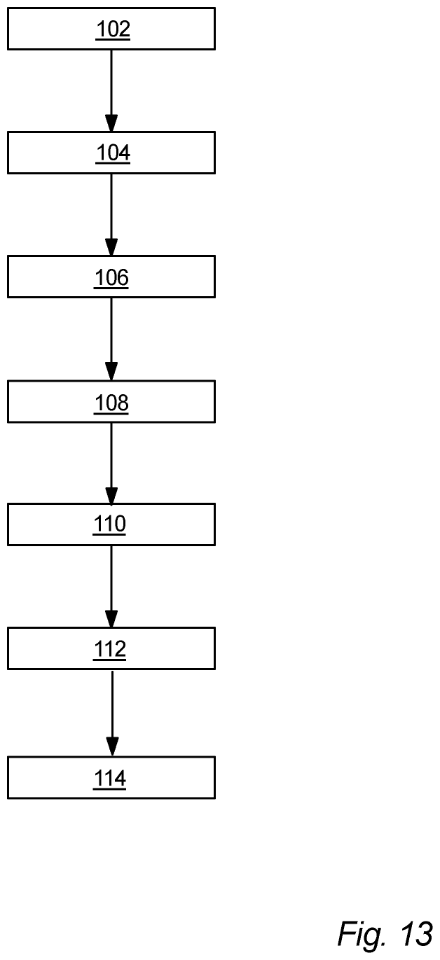

FIG. 13 shows an example of a processing method for processing of mycelium objects according to an embodiment of the invention. Initially, a mycelium object is provided 102, e.g. either as a piece of mycelium that has not been cut or trimmed in advance or as a mycelium object that may be at least one pre-cut piece of mycelium. The mycelium object is placed into a process chamber 104 and subjected to pressurized processing fluid 106. The pressure of the pressurized processing fluid is increased until it reaches a supercritical condition 108. It should be noted that the pressure may be increased further beyond the critical point and that furthermore, the temperature of the processing fluid may be controlled simultaneously to achieve a desired process. The mycelium object is subjected 110 to a reactive chemical agent such as a dyeing agent for a period of time. At the end of the processing period, the pressure is reduced 112 and the mycelium object is subsequently removed 114.

It should be noted in connection with process 110 of subjecting the mycelium object to the reactive chemical agent, that the reactive chemical agent may have been added to the pressurized fluid previously in connection with process 106 or in connection with process 108, and that even further, the reactive chemical agent may have been introduced to the process chamber before or at process 104.

In principle, the above method may be carried out at any stage of the mycelium processing procedure, but most advantageously, the supercritical conditions may be applied on the deacetylation DEP, plastification PP and/or dyeing processes DYP.

### FIGURE REFERENCES

30. Mycelium object
34. Process chamber
36. Controllable compressor
38. Storage container
40. Controller
42. Introducing member
44. Reducing member
46. Separator
48. Outlet
50. Residue outlet
52. Source of reactive chemical agent
54. Controllable inlet
56. Reactive chemical agent
60. Temperature sensor
62. Heater and/or cooler
70. Recirculation connection
72. Mass, thickness and/or volume detector
74. Supply of mycelium objects
80. Recirculation connection
82. Recirculation compressor
102. Providing a mycelium object
104. Mycelium object into process chamber
106. Subjecting to pressurized processing fluid
110. Mycelium object subjected to a reactive chemical agent
112. Reducing the pressure
114. Removing the mycelium object
A. Ambient
CP. Critical point
G. Gas
L. Liquid
P. Pressure
S. Solid
SCF. Supercritical fluid
T. Time
Tinc. Time of pressure increase
Tred. Time of pressure reduction
IPgrad-iI. Pressure increase gradient
IPgrad-rI. Pressure reduction gradient
Td. Time of subjecting to dyeing agent
SOP Soaking process
DEP Deacetylation process
PP Plastification process
DYP Dyeing process
FLP Fat liquoring process
MYP Mycelium product

1. A method of processing a mycelium object in the form of a mycelium fibril into a mycelium product,
the mycelium object comprising hyphae cells, said hyphae cells having cell walls, the cell walls of said hyphae cells comprising natural polymer including chitin/chitosan polymer,
the method of processing the mycelium object includes a reconstruction process for reconstructing mycelium objects in the form of mycelium fibrils into a mycelium fiber,
the method of processing the mycelium object further including at least one of the following processes:
a deacetylation process,
a plastification process,
a dyeing process, and
a fat liquoring process.

2. The method of processing a mycelium object according to claim 1,
the mycelium object comprising hyphae cells, said hyphae cells having cell walls, the cell walls of said hyphae cells comprising natural polymer including chitin/chitosan polymer,
wherein at least one of the processes,
the deacetylation process,
the plastification process,
the dyeing process, and/or
the fat liquoring process,
is performed by subjecting the mycelium object to an exposure of a reactive chemical agent and a pressurized processing fluid in a supercritical condition.

3. (canceled)

4. The method of processing a mycelium object according to claim 2,
wherein the method of processing a mycelium object into a mycelium product includes at least the two processes:
the plastification process, and
the dyeing process, wherein the method further includes
the fat liquoring process, and wherein
at least the plastification process and/or the dyeing process is performed by subjecting the mycelium object to an exposure of a reactive chemical agent and a pressurized processing fluid in a supercritical condition,
and wherein the fat liquoring process is performed under non-supercritical conditions.

5-7. (canceled)

8. The method of processing a mycelium object according to claim 2,
wherein the method further includes the process of pre-soaking the mycelium object prior to said exposure of a reactive chemical agent and a processing fluid in a supercritical condition.

9. The method of processing a mycelium object according to claim 2, wherein the method of processing the mycelium object by a pressurized processing fluid in a supercritical condition into a mycelium product includes the following processes:
a plastification process,
a dyeing process,
a fat liquoring process, and
a reconstruction process, and wherein at least the following processes are performed in the below consecutive order:
a plastification process,
a dyeing process, and
a fat liquoring process.

10-13. (canceled)

14. The method of processing a mycelium object according to claim 2,
wherein the plastification process and the dyeing process are performed simultaneously by subjecting the mycelium object to a crosslinking agent being able to bind covalently to chitosan amine groups, a dyeing agent being able to bind to chitin/chitosan hydroxyl groups and a pressurized processing fluid in a supercritical condition being able to efficiently penetrate the mycelium.

15. (canceled)

16. The method of processing a mycelium object according to claim 2,
wherein said processing fluid is/comprises carbon dioxide.

17-18. (canceled)

19. The method of processing a mycelium object according to claim 1,
wherein the chemical reactive agent comprises a crosslinking agent.

20. The method of processing a mycelium object according to claim 19, wherein said crosslinking agent is applied to facilitate covalent bonding between mycelium fibrils and/or mycelium fiber(s).

21-26. (canceled)

27. The method of processing a mycelium object according to claim 1, wherein the reactive chemical agent comprises a dyeing agent.

28-32. (canceled)

33. The method of processing a mycelium object according to claim 1, wherein the percentage of chitosan amine groups covalently bound to said plastification agent is above 50%.

34. The method of processing a mycelium object according to claim 1, wherein the dyeing agent is able to bind covalently to chitin hydroxyl groups and/or chitosan amine groups.

35-43. (canceled)

44. The method of processing a mycelium object according to claim 1, wherein the mycelium object processed is a filament or yarn reconstructed on the basis of fibrils of chitinous polymer.

45. The method of processing a mycelium object according to claim 2, wherein the method includes a plastification process and/or a dyeing process said method comprising the following processes:
providing a mycelium object,
introducing the mycelium object into a process chamber,
subjecting the mycelium object in the process chamber to a pressurized fluid,
controlling the pressure of the pressurized fluid to reach supercritical conditions,
subjecting the mycelium object to a reactive chemical agent, which reactive chemical agent is dissolved and/or transported in the pressurized fluid for at least a predefined period of time while the pressurized fluid is in supercritical conditions,
reducing the pressure in the process chamber, and
removing the mycelium object from the process chamber,
wherein the process of reducing the pressure in the process chamber comprises controlling the pressure reduction over time.

46. (canceled)

47. A reconstructed filament wherein the reconstructed filament comprises mycelium fibrils and an additive.

48-49. (canceled)

50. The reconstructed filament according to claim 47, wherein the mycelium fibrils originate from mechanically subdivided natural mycelium.

51-60. (canceled)

61. The reconstructed filament according to claim 47, wherein the filament comprises fibrils between 0.01 and 30 wt. % of a further fibril type of at least one of the following natural fibrous proteins: Collagen, elastin, keratin, and resilin, and/or cellulosic fibrils originating from other sources than mycelium.

62. (canceled)

63. The reconstructed staple fiber manufactured on the basis of a reconstructed filament according to claim 47.

64. A wool comprising of a plurality of staple fibers made of the reconstructed filament according to claim 47, wherein the natural mycelium fibrils originate from mechanically subdivided natural mycelium fibers.

65. (canceled)

66. A yarn spun from staple fibers of reconstructed filament according to claim 63.

67. (canceled)
